(19) United States
(12) Patent Application Publication
(10) Pub. No.: US 2013/0217657 A1
(43) Pub. Date: Aug. 22, 2013

(54) COMBINATION ANTI-INFLAMMATORY OPHTHALMIC COMPOSITIONS

(71) Applicant: INSITE VISION INCORPORATED, (US)

(72) Inventors: Richard L. Lindstrom, Wayzata, MN (US); Kamran Hosseini, Hayward, CA (US); Lyle M. Bowman, Pleasanton, CA (US); Sui Yen Eddie Hou, Foster City, CA (US)

(73) Assignee: INSITE VISION INCORPORATED, Alameda, CA (US)

(21) Appl. No.: 13/849,837

(22) Filed: Mar. 25, 2013

Related U.S. Application Data
(63) Continuation-in-part of application No. 13/333,534, filed on Dec. 21, 2011.

(51) Int. Cl. A61K 31/573; A61K 31/407; A61K 45/06; A61K 31/57; A61K 31/56; A61K 31/196; A61K 31/165

(52) U.S. Cl. CPC A61K 31/573; A61K 31/196; A61K 31/407; A61K 31/165; A61K 31/57; A61K 31/56; A61K 45/06
USPC 514/171

(57) ABSTRACT

Compositions and systems for topical ophthalmic application, which include an aqueous mixture of steroidal and non-steroidal anti-inflammatory agents in a flowable mucoadhesive polymer, for treating inflammation and inflammatory conditions of the eye.

### RELATED APPLICATIONS

This application is a Continuation in Part of U.S. application Ser. No. 13/333,534, filed on Dec. 21, 2011, the disclosure of which Application is incorporated by reference herein.

### FIELD

The present disclosure relates to ophthalmic formulations; more particularly to ophthalmic formulations employing combined non-steroidal anti-inflammatory and steroidal anti-inflammatory agents.

### BACKGROUND

A variety of diseases and disorders of the eye are associated with and/or indicated by inflammation of the eye including but not limited to, scleritis, episcleritis, dry eye, blepharitis, conjunctivitis, and uveitis, including iritis, cyclitis, retinitis, and choroiditis. Inflammation of the eye can also occur due to trauma to the eye or post-operatively, such as after cataract surgery or laser surgery, for example.

Treatment of inflammation of the eye can involve frequent dosing regimens which can erode patient compliance. From a delivery perspective, further challenges include formulating ophthalmic vehicles at viscosities low enough for reliable administration in drop form without negatively influencing delivery efficiency and, at the same time, maintaining sufficient viscosity and mucoadhesion so that the delivered medicament remains in or on the ocular surface for a sufficient period of time to effectively treat the inflamed eye. Drug delivery to the ocular surface and mucosa faces the additional obstacle of various clearance mechanisms present in the eye.

### SUMMARY

The present disclosure provides an ophthalmic composition comprising a therapeutically effective amount of a non-steroidal anti-inflammatory agent (NSAID), a therapeutically effective amount of a steroidal anti-inflammatory, and an ophthalmically acceptable vehicle comprising a flowable mucoadhesive polymer. The composition has a viscosity formulated for administration to the eye of a mammal in drop form. The flowable mucoadhesive polymer may be a lightly cross-linked carboxy-containing polymer. The ophthalmically acceptable vehicle may further comprise chitosan, and in certain embodiments the chitosan is in sufficient concentration to allow the flowable mucoadhesive polymer to remain in suspension. The NSAID may be present in a range from about 0.001% to about 1.0% by weight of the composition.

In some embodiments, the NSAID may be selected from the group consisting of: bromfenac, ketorolac, or nepafenac. In some compositions, the NSAID may be nepafenac and the glucocorticoid may be dexamethasone. The steroidal anti-inflammatory may be present in a range from about 0.01% to about 1% by weight of the composition. The composition may have a pH of about 6.0 to about 8.6.

The ophthalmic composition may have a viscosity in the range of about 1,000 to about 30,000 cps, for example, from about 1,000 to about 5,000 cps. The composition may further comprise an additional therapeutically active agent selected from the group consisting of antibacterial antibiotic agent, synthetic antibacterial agent, antifungal antibiotic agent, synthetic antifungal agent, antineoplastic agent, anti-allergic agent, glaucoma-treating agent, antiviral agent and anti-mycotic agent.

In accordance with another aspect of the inventive concept, provided is a method for therapeutic treatment of an inflammatory condition of the eye or surrounding tissue in a mammal comprising the steps of: (a) providing a therapeutically effective amount of a non-steroidal anti-inflammatory agent (NSAID), a therapeutically effective amount of a steroidal anti-inflammatory, and an ophthalmically acceptable vehicle comprising a flowable mucoadhesive polymer, wherein the composition has a viscosity formulated for administration to the eye of a mammal in drop form; and (b) administering said composition to the eye of a mammal to treat inflammation or inflammatory conditions of the eye and/or surrounding tissue. The inflammatory condition may be a retinal condition selected from the group consisting of: age related macular degeneration, AIDS-related ocular disease, CMV retinitis, birdshot retinochoroidopathy (BR), choroidal melanoma, coats disease, cotton wool spots, diabetic retinopathy diabetic macular edema, cystoid macular edema, lattice degeneration, macular disease, macular degeneration, hereditary macular dystrophy, macular edema, macular hole, macular pucker, central serous chorioretinopathy, ocular histoplasmosis syndrome (OHS), posterior vitreous detachment, retinal detachment, retinal artery obstruction, retinal vein occlusion, retinoblastoma, retinopathy of prematurity (ROP), retinitis pigmentosa, retinoschisis (acquired and x-linked), stargardt's disease, toxoplasmosis of retina or uveitis. The inflammatory condition may be a cystoid macular edema.

It is to be understood that both the foregoing general description and the following detailed description are exemplary and explanatory only and are not restrictive of the invention, as claimed.

### DETAILED DESCRIPTION

The present disclosure provides topical ophthalmic formulations containing a non-steroidal anti-inflammatory agent (NSAID) and a steroidal anti-inflammatory agent such as dexamethasone, prednisone, fluoromethalone, loteprednol etabonate, or difluprednate in an ophthalmically acceptable vehicle. The combination therapy enables control of inflammation via use of reduced dosages of each of the individual anti-inflammatory components, compared to typical dosing of a single agent, thus reducing the side effects of each agent. For example, typical dosing of ophthalmic steroidal agents alone can result in elevated intraocular pressure (TOP) and can slow healing of wounds in the eye. Typical dosing regimens of NSAIDs, on the other hand, can cause keratitis. In some patients, NSAID usage can result in epithelial breakdown, corneal thinning, erosion, ulceration, and/or perforation. The side effects due to either agent alone can reduce the period of time over which the drug can be administered, while inflammation, or the underlying cause of inflammation, may persist.

NSAIDs are known to inhibit cyclooxygenases, enzymes associated with pain and inflammation in mammals. Cyclooxygenases are essential in the biosynthesis of prostaglandins, which have been shown in many animal models to be mediators of intraocular inflammation. Although steroidal compounds have been used to treat such inflammation, NSAIDs from the group of drugs known as cyclooxygenase inhibitors have been substituted for steroids because they have not shown the same propensity to produce side-effects in ocular tissues as compared to ophthalmic steroids. Non-steroidal agents are also widely prescribed to reduce pain and inflammation in a wide number of tissues. When used as topical agents in the eye, they suppress inflammatory responses and have been shown to prevent particular side-effects of surgical trauma (on the pupil preventing surgical meiosis), fluid accumulating in the back of the eye after cataract surgery (post-surgical macular edema) and the appearance of inflammatory cells and vessel leakage in the anterior chamber. Topical application of non-steroidal anti-inflammatory agents in the eye also appears to relieve some of the itching due to allergic conjunctivitis. Diclofenac sodium, suprofen, and flurbiprofen are non-steroidal anti-inflammatory agents that have been used for the treatment of postoperative inflammation in patients who have undergone cataract extraction.

As used herein the term “ophthalmic composition” refers to a composition intended for application to the eye and/or its related and/or surrounding tissues such as, for example, eyelid. The term also includes compositions intended to therapeutically treat conditions of the eye itself or the tissues surrounding the eye and compositions administered via the ophthalmic route to treat therapeutically a local condition other than that involving the eye. The ophthalmic composition can be applied topically and to the eye or surrounding tissue or by other techniques, known to persons skilled in the art, such as injection to the eye or its related tissues or direct application to the tissue. Examples of suitable topical administration to the eye include administration of eye drops and by spray formulations. A further suitable topical administration route is by subconjunctival injection. The agents can also be provided to the eye periocularly or retro-orbitally. Although it is an advantage of the invention that intracameral administration is not required, this and other routes of administration are not outside the scope of the invention.

As used herein an “ophthalmically acceptable vehicle” is one which allows delivery of a medicament to the eye and/or eyelids and/or surrounding tissue, to treat an ocular disease or condition without significant deleterious effects on the eye. An ophthalmically acceptable vehicle is one that can maintain proper intraocular pressure and provide solutions of medicaments that are isotonic, mildly hypotonic, or mildly hypertonic. To maintain such conditions one can include various non-ionic osmolality-adjusting compounds such as polyhydric alcohols, including for example, glycerol, mannitol, dextrose, sorbitol, or propylene glycol. Alternatively, osmolality adjusting compounds can include ionic salts such as sodium or potassium chloride. An ophthalmically acceptable vehicle can also include buffers to adjust the vehicle to an acceptable pH, which can range from about 3 to 6.5, and in some embodiments from about 4 to 9, including any pH in between. Compositions of the present disclosure can have a pH at the upper end of this scale as described herein. Such buffer systems include, but are not limited to, acetate buffers, citrate buffers, phosphate buffers, borate buffers and mixtures thereof. Specific buffer components useful in the present application include, but are not limited to, citric acid/sodium citrate, boric acid, sodium borate, sodium phosphates, including mono, di- and tri-basic phosphates, such as sodium phosphate monobasic monohydrate and sodium phosphate dibasic heptahydrate, tromethanime base and hydrochloride and mixtures thereof. It should be noted that any other suitable ophthalmically acceptable buffer components can be employed to maintain the pH of the ophthalmic formulation so that the ophthalmic formulation is provided with an acceptable pH, and the foregoing buffer components are merely exemplary examples of such buffer components.

As used herein “an ophthalmically acceptable salt” includes those that exhibit no significant deleterious effects on the eye as well as being compatible with the active ingredient itself and the components of the ophthalmically acceptable vehicle. Salts or zwitterionic forms of a medicament can be water or oil-soluble or dispersible. The salts can be prepared during the final isolation and purification of the medicament or separately by adjusting the pH of the appropriate medicament formulation with a suitable acid or base. An ophthalmically acceptable salt can also include the aforementioned buffer systems.

As used herein, the term “carboxyl-containing polymer” refers to a polymer that contains the carboxylic acid functional group. This functional group can be substantially ionized, for example, and exist as a carboxylate anion (COO⁻), rendering the polymer negatively charged. In the context of an ophthalmically acceptable vehicle, the degree of ionization can depend on the pH, which is mediated by any buffer system, and the presence of other components in the vehicle that contain Lewis basic atoms, such as an amine-functionalized polymer. A Lewis base is donor of a pair of electrons and as such, is capable of accepting hydrogen ion (H⁺) from a carboxyl group (COOH).

As used herein, the term “cationic polymer” refers to a positively-charged, amine-functionalized polymer. The polymer contains nitrogen atoms that are quaternized or capable of being quaternized upon adjustment to a sufficiently low pH and/or in the presence of a proton donor, such as the carboxyl containing polymer, or other Lewis acid (i.e. an electron pair acceptor). A quaternized nitrogen atom is a nitrogen atom engaged in bonding to four other atoms, thus causing nitrogen to have a net formal charge of plus one (+1). Examples of nitrogen atoms carrying positive charge include, but not limited to, NR₄⁺, NR₃H⁺, NR₂H⁺, NRH₂⁺, wherein R can represent any atom or group of atoms bonded to nitrogen.

As used herein “viscosity” refers to a fluid's resistance to flow. The unit of viscosity is dyne second per square centimeter [dyne·s/cm²], or poise [P]. This type of viscosity is also called dynamic viscosity, absolute viscosity, or simple viscosity. This is distinguished from kinematic viscosity which is the ratio of the viscosity of a fluid to its density.

As used herein “mucoadhesive” or “mucoadhesion” refers to the ability of the ophthalmically acceptable vehicle to adhere to the ocular mucosa. Mucoadhesive agents used in the disclosure include carboxy-containing polymers capable of forming hydrogen bonds. Mucoadhesion can depend on pH and the density of hydrogen bonding groups. In the vehicle of the present invention, the density of cross-linking in the carboxy-containing polymer can affect mucoadhesion. Thus, a lightly cross-linked polymer system has sufficient flexibility to form multiple hydrogen bonds, making it a good mucoadhesive agent. Another vehicle component that can affect mucoadhesion is the presence of a secondary polymer, which can interact with the carboxy-containing polymer, as explained further below.

As used herein the term “flowable mucoadhesive polymer” refers to a carboxy-containing polymer, e.g., lightly crosslinked polymers of acrylic acid or the like, having an optimal in vivo mucosal absorption rate, safety, degradability and flowability for an eye drop. The flowable mucoadhesive polymers used in the present disclosure are water insoluble, water-swellable, biodegradable polymer carriers including lightly crosslinked carboxy-containing polymers such as polycarbophil (Noveon® AA-1, Lubizol Corp., Wickliffe, Ohio) or other Carbopol® polymers (Lubizol Corp., Wickliffe, Ohio). Suitable carboxy-containing polymers for use in the present invention and methods for making them are described in U.S. Pat. Nos. 5,192,535 to Davis et al. which is hereby incorporated in its entirety by reference. A suitable carboxy-containing polymer system for use in the present invention is known by the tradename DuraSite® (InSite Vision Inc., Alameda, Calif.), containing polycarbophil, which is a sustained release topical ophthalmic delivery system that releases drug at a controlled rate. DuraSite® encompasses lightly crosslinked polymers that are prepared by suspension or emulsion polymerizing at least about 90% by weight of a carboxyl-containing monoethylenically unsaturated monomer such as acrylic acid with about 0.1% to about 5% by weight of a polyfunctional, or difunctional, crosslinking agent such as divinyl glycol (3,4-dihydroxy-1,5-hexadiene), having a particle size of not more than about 50 μm in equivalent spherical diameter.

As used herein the term “lightly crosslinked polymer” encompasses any polymer prepared by suspension or emulsion polymerization having a main polymer backbone comprising at least about 90% by weight of the polymer with a crosslinking agent present in a range from about 0.1% to about 5% by weight of the polymer, including about 0.1%, about 0.2%, about 0.3%, about 0.5%, about 0.6%, about 0.7%, about 0.8%, about 0.9%, about 1.0%, about 1.5%, about 2.0%, about 2.5%, about 3.0%, about 3.5%, about 4.0%, about 4.5%, and about 5.0%, including any fractional amount in between. In some embodiments, the main polymer backbone comprises from about 90% to about 99.9% by weight of the polymer. In some embodiments, the main polymer backbone comprises about 90%, about 91%, about 92%, about 93%, about 94%, about 95%, about 96%, about 97%, about 98%, about 99%, about 99.5%, or about 99.9% by weight of the polymer, including any fractional amount in between. The main polymer backbone can comprise a single monomer unit or can be a copolymer comprising two, three, or any number of monomer units. At least one monomer unit of a main polymer backbone has a functional moiety capable of supporting a charge, such as a carboxyl group, a sulfate group, a phosphate group, and the like. The crosslinking agent may be any difunctional or polyfunctional crosslinking agent.

When formulated with an ophthalmic medicament, e.g., an NSAID in combination with a glucocorticoid, such as dexamethasone, into solutions or suspensions in aqueous medium, the amount of lightly crosslinked polymer ranges from about 0.5% to about 1.5% by weight, based on the total weight of the aqueous suspension. The pH is from about 6.0 to about 8.6 and the osmotic pressure (osmolality or tonicity) is from about 10 mOsM to about 400 mOsM. Such formulations provide new topical ophthalmic medicament delivery systems having suitably low viscosities which permit them to be easily administered to the eye in drop form, and hence be comfortably administrable in consistent, accurate dosages. The compositions containing DuraSite® rapidly gel in the eye after coming into contact with the eye's tear fluid to a substantially greater viscosity than that of the originally-introduced suspension or solution and thus remain in place for prolonged periods of time to provide sustained release of the ophthalmic medicament.

As used herein, “administered to the eye” means that an ophthalmically acceptable vehicle, along with a medicament, is in the form of an eye drop that can be applied directly to the surface of the eye and/or in the eyelid margins, such administration techniques being familiar to persons skilled in the art.

As used herein the term “retained in or carried with” or “retaining or carrying” embraces generally all ways that the steroidal and nonsteroidal agents can be associated with the flowable mucoadhesive polymer. For example, they can be in aqueous solution dispersed throughout the polymer. A solution of an NSAID having a concentration of 0.01% up to about 2.0% can be mixed with or dispersed throughout the flowable mucoadhesive polymer carrier. An NSAID can also be in suspension with the polymer depending on its concentration. For example, when bromfenac is used in an amount more than about 9.0% by weight of the composition, some of the bromfenac can be in suspension with the polymer carrier while some of the it will still be in solution and mixed with the polymer carrier. In the case of nepafenac, if the concentration is at 0.1% or greater, some of the drug will be in solution and some in suspension. In the case of nepafenac, the formulation is a suspension at 0.1%. The formulation is dependent on the specific therapeutic compound and can be formulated to be in solution, suspension and both depending the drug concentration.

As used herein the term “inflammation or inflammatory conditions of the eye” refers to an ocular disease or any inflammatory condition of the eye and external tissues surrounding eye, e.g., eyelid, meibian glands, etc. influenced by various exogenous or endogenous agents or events. Endogenous factors include, but are not limited to, inflammatory chemokines, cytokines, mediators, nuclear transcription factors, antigens, autogens or hormones that can cause acute or chronic inflammation, pain, redness, swelling, watering or tearing and itchiness of the eye or its surrounding tissues. Exogenous agents or events include, but are not limited to, infection, injury, allergies, radiation, surgery or damage to the eye or its surrounding tissues, which initiate biochemical reactions leading to an inflammation. An ocular disease is one caused by vascular leakage in the eye or inflammation in the eye. Examples of conditions related to inflammation in the eye include, but are not limited to the following: surgical trauma; dry eye; allergic conjunctivitis; viral conjunctivitis; bacterial conjunctivitis; blepharitis; anterior uveitis; injury from a chemical; radiation or thermal burn; or penetration of a foreign body, signs and symptoms of eye problems (e.g., pain in or around the eye, redness especially accompanied by pain in the eye (with or without movement), extreme light sensitivity, halos (colored circles or halos around lights), bulging (protrusion) of the eye or swelling of eye tissues, discharge, crusting or excessive tearing; eyelids stuck together, especially upon awakening, blood inside the front of the eye (on the colored part) or white of the eye); cataracts; pain and inflammation associated with wearing contact lenses; corneal conditions (e.g., conjunctival tumor excision, conjunctivitis (“Pink Eye”), cornea edema after cataract surgery, corneal clouding, corneal transplantation, corneal ulcer, dry eye syndrome, dystrophies, conditions associated with excimer laser phototherapeutic keratectomy, herpes simplex keratitis, keratoconus, pterygium, recurrent erosion syndrome); eye movement disorders; glaucoma; ocular oncology, oculoplastics (e.g., cosmetic surgery, enucleation, eyelid and orbit injuries, ectropion, entropion, Graves' disease, involuntary eyelid blinking); conditions associated with refractive surgery; and retinal conditions.

As used herein the term “sustained release delivery system” or “sustained release composition” refers to a composition comprising a flowable mucoadhesive polymer—which is a carboxy-containing polymer such as polycarbophil and DuraSite®, as described in U.S. Pat. No. 5,192,535—which facilitates a sustained release of the combination steroidal and NSAID agents. Such compositions may include other biologically active agents in addition to the NSAID and steriodal anti-inflammatory combination. Typically, the present sustained release compositions can contain from about 0.005% (w/w) to about 0.5% of NSAID. In an exemplary embodiment, the range of the NSAID loading can be in a range from about 0.001% (w/w) to about 1.0. The sustained release delivery systems or compositions of this disclosure can be formed into many shapes such as a solution, a gel, a film, a pellet, a rod, a filament, a cylinder, a disc, a wafer, nanoparticles or a microparticle. A “microparticle” as defined herein, comprises a blend polymer component having a diameter of less than about one millimeter and having bromfenac dispersed therein. A microparticle can have a spherical, non-spherical or irregular shape. Typically, the microparticle will be of a size suitable for comfortable topical application to the eye. In one embodiment, the size range for microparticles is from about one to about 25 microns in diameter.

As defined herein, a sustained release of a biologically active agent is a release of the biologically active agent(s), such as a combination of NSAID and glucocorticoid, from a sustained release delivery system or composition. The release occurs over a period which is longer than that period during which a therapeutically significant amount of the biologically active agent would be available following direct administration of a solution of the biologically active agent. In one embodiment, a sustained release occurs over a period of greater than six to twelve hours such as about twenty-four hours or longer. A sustained release of biologically active agent(s) can be a continuous or a discontinuous release, with relatively constant or varying rates of release. The continuity of release and level of release can be manipulated by the type of polymer composition used (e.g., monomer ratios, molecular weight, and varying combinations of polymers), agent loading, and/or selection of excipients to produce the desired effect.

As used herein the term “treating” or “treatment” refers to reducing, ameliorating reversing, alleviating, inhibiting the progress of, or preventing or slowing down progression or onset of a disease or a medical condition of the eye itself or the tissue surrounding the eye or the symptoms associated therewith. The term also encompasses prophylaxis, therapy and cure. The subject receiving “treatment,” or whom undergoes “treating” is any mammal in need of such treatment for (eye-related inflammation or inflammatory conditions), including primates, in such as humans, and other mammals such as equines, cattle, swine and sheep; and poultry and domesticated mammals and pets in general.

The term “therapeutically effective amount” as used herein means that the amount of a composition elicits a beneficial biological or medicinal response in a tissue, system, animal or human. For example, a therapeutically effective amount of a composition of the disclosure is a dose which leads to a clinically detectable improvement or treatment (as defined above) of the eye of a subject suffering from an inflammatory eye condition or disease. An “effective amount” when used in connection with treating an ocular disease or condition is intended to qualify the amount of a medicament used in the treatment of a particular ocular disease or condition. This amount will achieve the goal of preventing, reducing, or eliminating the ocular disease or condition. An effective amount depends on the particular active ingredient to be administered, although ophthalmic formulations can include, for example, from about 0.001% to about 5.0% by weight, while in other embodiments the active ingredient is present in a range from about 0.08% to about 0.12% by weight. 0.01 mg/ml to 100 mg/ml per dose in one embodiment and from about 10 to 50 mg/ml dose in another embodiment. An “effective amount” can include a dose regimen once per day, twice per day, thrice per day, or intermittently during treatment, and so on.

As used herein, the term “about” refers to an approximation of a stated value within an acceptable range, such as plus or minus about 5% of the stated value.

Retinal conditions include, but are not limited to, age related macular degeneration, AIDS-related ocular disease (e.g., CMV retinitis), birdshot retinochoroidopathy (BR), choroidal melanoma, coats disease, cotton wool spots, diabetic retinopathy, diabetic macular edema, cystoid macular edema, lattice degeneration, macular disease (e.g., macular degeneration, hereditary macular dystrophy, macular edema, macular hole, macular pucker, central serous chorioretinopathy), ocular histoplasmosis syndrome (OHS), posterior vitreous detachment, retinal detachment, retinal artery obstruction, retinal vein occlusion, retinoblastoma, retinopathy of prematurity (ROP), retinitis pigmentosa, retinoschisis (acquired and x-linked), stargardt's disease, toxoplasmosis (affecting retina) and uveitis.

Bromfenac is a non-steroidal anti-inflammatory agent which has the structural formula of

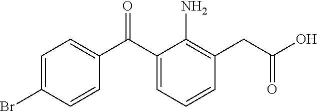 [SMILES: Nc1c(CC(=O)O)cccc1C(=O)c1ccc(Br)cc1]

Nepafenac is a is a non-steroidal anti-inflammatory agent which has the structural formula of

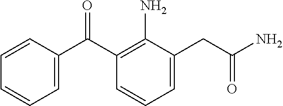 [SMILES: NC(=O)Cc1cccc(C(=O)c2ccccc2)c1N]

Ketorolac is a non-steroidal anti-inflammatory agent which has the structural formula of

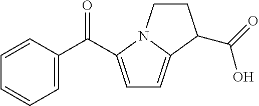 [SMILES: O=C(c1ccccc1)c1ccc2n1CCC2C(=O)O]

The above compounds and other NSAIDS to be used in accordance with the disclosure may be in a salt form or a hydrated form or both. The salt forms include alkali metal salts such as sodium salt and potassium salt, alkaline earth metal salts such as calcium salt and magnesium salt, among others, and any salt may suitably be used, provided that it can attain the object of the inventive concept. The hydrated forms include monohydrate, sessquihydrate (1.5 H₂O), dihydrate, pentahydrate and any other hydrate forms may suitably be used, provided that it can attain the object of the inventive concept.

Glucocorticoids can initiate an anti-inflammatory effect by binding to the cytosolic glucocorticoid receptor (GR). After binding GR, the receptor-ligand complex translocates to the cell nucleus, where it can bind to glucocorticoid response elements (GRE) in the promoter region of target genes. The proteins encoded by these upregulated genes have a wide range of effects including anti-inflammatory effects mediated, for example, by lipocortin I as described above. These glucocorticoids can also reduce the transcription of pro-inflammatory genes by a mechanism of transrepression. Thus, inflammation associated with blepharitis or other optical conditions can be ameliorated by glucocorticoid treatment.

Accordingly, in some embodiments, the compositions may be formulated to include steroid anti-inflammatory in addition to dexamethasone, prednisone, fluoromethalone, loteprednol etabonate, or difluprednate. Such steroidal anti-inflammatory agents are selected from for example, hydrocortisone, cortisone acetate, prednisone, prednisolone, methylprednisolone, dexamethasone, betamethasone, triamcinolone, beclomethasone, and fluorometholone. In some embodiments, the glucocorticoids include, for example, 21-acetoxypregnenolone, alclometasone, algestone, amcinonide, budesonide, chloroprednisone, clobetasol, clobetasone, clocortolone, cloprednol, corticosterone, cortisone, cortivazol, deflazacort, desonide, desoximetasone, diflorasone, diflucortolone, difluprednate, enoxolone, fluazacort, flucloronide, flumethasone, flunisolide, fluocinolone acetonide, fluocinonide, fluocortin butyl, fluocortolone, fluoromethalone, fluperolone acetate, fluprednidene acetate, fluprednisolone, flurandrenolide, fluticasone propionate, formocortal, halcinonide, halobetasol propionate, halometasone, halopredone acetate, hydrocortarnate, loteprednol etabonate, mazipredone, medrysone, meprednisone, mometasone furoate, paramethasone, prednicarbate, prednisolone 25-diethylamino-acetate, prednisolone sodium phosphate, prednival, prednylidene, rimexolone, tixocortol, triamcinolone acetonide, triamcinolone benetonide, triamcinolone hexacetonide, difluprednate their opthalmically acceptable salts, combinations thereof, and mixtures thereof. In certain embodiments, the glucocorticoid includes dexamethasone, prednisone, prednisolone, methylprednisolone, medrysone, triamcinolone, loteprednol etabonate, or ophthalmically acceptable salts thereof, and combinations thereof.

The present disclosure also provides kits including a composition having a NSAID and steriodal anti-inflammatory, such as for example, bromfenac and dexamethasone in an ophthalmic carrier comprising a flowable mucoadhesive polymer for application to the eye of a mammal. The kit may further include instructions for how to use the composition, an eye dropper and/or other useful paraphanalia for topical delivery to the eye. The kit can provide the active anti-inflammatory agents as solids with a sterile aqueous solution to mix real time, or can provide the agents pre-mixed in the carrier vehicle.

The lightly crosslinked polymers of acrylic acid or the like used in practicing this disclosure are, in general, well known in the art. In one embodiment such polymers are ones prepared from at least about 90% or from about 95% to about 99.9% by weight, based on the total weight of monomers present, of one or more carboxyl-containing monoethylenically unsaturated monomers. Acrylic acid is a carboxyl-containing monoethylenically unsaturated monomer, but other unsaturated, polymerizable carboxyl-containing monomers, such as methacrylic acid, ethacrylic acid, beta-methylacrylic acid (crotonic acid), cis-alpha-methylcrotonic acid (angelic acid), trans-alpha-methylcrotonic acid (tiglic acid), alpha.-butylcrotonic acid, alpha-phenylacrylic acid, alpha-benzylacrylic acid, alpha-cyclohexylacrylic acid, beta-phenylacrylic acid (cinnamic acid), coumaric acid (o-hydroxycinnamic acid), umbellic acid (p-hydroxycoumaric acid), and the like can be used in addition to or instead of acrylic acid.

Such polymers are crosslinked by using a small percentage, i.e., less than about 5%, such as from about 0.5% or from about 0.1% to about 5%, or from about 0.2% to about 1%, based on the total weight of monomers present, of a polyfunctional crosslinking agent. Included among such crosslinking agents are non-polyalkenyl polyether difunctional crosslinking monomers such as divinyl glycol; 2,3-dihydroxyhexa-1,5-diene; 2,5-dimethyl-1,5-hexadiene; divinylbenzene; N,N-diallylacrylamide; N,N-diallylmethacrylamide and the like. Also included are polyalkenyl polyether crosslinking agents containing two or more alkenyl ether groupings per molecule, or alkenyl ether groupings containing terminal H₂C═C< groups, prepared by etherifying a polyhydric alcohol containing at least four carbon atoms and at least three hydroxyl groups with an alkenyl halide such as allyl bromide or the like, e.g., polyallyl sucrose, polyallyl pentaerythritol, or the like; see, e.g., Brown U.S. Pat. No. 2,798,053, which incorporated herein by reference in its entirety. Diolefinic non-hydrophilic macromeric crosslinking agents having molecular weights of from about 400 to about 8,000, such as insoluble di- and polyacrylates and methacrylates of diols and polyols, diisocyanate-hydroxyalxyl acrylate or methacrylate reaction products, and reaction products of isocyanate terminated prepolymers derived from polyester diols, polyether diols or polysiloxane diols with hydroxyalkylmethacrylates, and the like, can also be used as the crosslinking agents; see, e.g., Mueller et al. U.S. Pat. Nos. 4,192,827 and 4,136,250, which incorporated herein by reference in its entirety.

The lightly crosslinked polymers can of course be made from a carboxyl-containing monomer or monomers as the sole monoethylenically unsaturated monomer present, together with a crosslinking agent or agents. They can also be polymers in which up to about 40%, or from about 0% to about 20% by weight, of the carboxyl-containing monoethylenically unsaturated monomer or monomers has been replaced by one or more non-carboxyl-containing monoethylenically unsaturated monomers containing only physiologically and ophthalmologically innocuous substituents, including acrylic and methacrylic acid esters such as methyl methacrylate, ethyl acrylate, butyl acrylate, 2-ethylhexylacrylate, octyl methacrylate, 2-hydroxyethyl-methacrylate, 3-hydroxypropylacrylate, and the like, vinyl acetate, N-vinylpyrrolidone, and the like; see Mueller et al. U.S. Pat. No. 4,548,990, which incorporated herein by reference in its entirety, for a more extensive listing of such additional monoethylenically unsaturated monomers. In one embodiment, polymers are lightly crosslinked acrylic acid polymers wherein the crosslinking monomer is 2,3-dihydroxyhexa-1,5-diene or 2,3-dimethylhexa-1,5-diene.

The lightly crosslinked polymers disclosed herein are prepared by suspension or emulsion polymerizing the monomers, using conventional free radical polymerization catalysts, to a dry particle size of not more than about 50 μm in equivalent spherical diameter; e.g., to provide dry polymer particles ranging in size from about 1 to about 30 μm, or from about 3 to about 20 μm, in equivalent spherical diameter. In general, such polymers will range in molecular weight estimated to be about 250,000,000 to about 4,000,000,000 or about 500,000 to about 2,000,000,000 dalton

According to any of the above aspects the composition of the disclosure is an aqueous mixture that can also contain amounts of suspended lightly crosslinked polymer particles ranging from about 0.5% to about 1.5% by weight, or from about 0.8% to about 1.0% by weight, based on the total weight of the aqueous mixture. The aqueous mixture can be an aqueous solution of NSAID and a flowable mucoadhesive polymer or an aqueous suspension of NSAID and a flowable mucoadhesive polymer or a mixture of an aqueous solution and suspension of NSAID and a flowable mucoadhesive polymer. In certain embodiments, the composition is prepared using pure, sterile water, such as deionized or distilled, having no physiologically or ophthalmologically harmful constituents, and is adjusted to a pH of from about 7.4 to about 8.6, in some embodiments from about 8.2 to about 8.4, and in other embodiments to a pH of about 8.3 using any physiologically and ophthalmologically acceptable pH adjusting acid, base or buffer, e.g., acids such as acetic, boric, citric, lactic, phosphoric, hydrochloric, or the like, bases such as sodium hydroxide, sodium phosphate, sodium borate, sodium citrate, sodium acetate, sodium lactate, THAM (trishydroxymethylamino-methane), TRIS (tromethamine base) or the like and salts and buffers such as citrate/dextrose, sodium bicarbonate, ammonium chloride and mixtures of the aforementioned acids and bases. For example, the NSAID or its salt at may be dissolved and added by sterile filtration to a preparation containing sodium chloride, DuraSite® and surfactant. This mixture may then be adjusted to the appropriate pH by known techniques, for example by the addition of sodium hydroxide. Other methods will be apparent to one skilled in the art.

When formulating the composition as either an aqueous solution or an aqueous suspension, the osmolality can be adjusted to from about 10 mOsm/kg to about 400 mOsm/kg, using appropriate amounts of physiologically and ophthalmologically acceptable salts. Sodium chloride approximates physiologic fluid, and amounts of sodium chloride ranging from about 0.01% to about 1% by weight, or from about 0.05% to about 0.45% by weight, based on the total weight of the aqueous suspension, provide osmolalities within the above-stated ranges. Equivalent amounts of one or more salts made up of cations such as potassium, ammonium and the like and anions such as chloride, citrate, ascorbate, borate, phosphate, bicarbonate, sulfate, thiosulfate, bisulfate, sodium bisulfate, ammonium sulfate, and the like can also be used in addition to or instead of sodium chloride to achieve osmolalities within the above-stated ranges. Sugars like mannitol, dextrose, glucose or other polyols may be added to adjust the osmolality.

The amounts of flowable mucoadhesive polymer, the pH, and the osmotic pressure chosen from within the above-stated ranges are correlated with one another and with the degree of crosslinking of the polymer to give aqueous solutions or suspensions having viscosities ranging from about 1,000 to about 2,000 or 5,000 to about 20,000 cps respectively, as measured at room temperature (about 25° C.) using a Brookfield Digital LVT Viscometer equipped with a number 25 spindle and a 13R small sample adapter at 12 rpm. The compositions of the present disclosure have a viscosity that is suited for the selected route of administration. Alternatively, the viscosity can be 1000 to 5000 cps as measured with a Brookfield cone and plate viscosity DV-II+ with the spindle No. CP-52 at 6 rpm.

Compositions delivered by means of the sustained release medicament delivery system of this disclosure typically have residence times in the eye ranging from about 4 to about 8 hours. The NSAID contained in these compositions is released from the composition at rates that depend on such factors as the NSAID itself and its physical form, the extent of drug loading and the pH of the system, as well as on any drug delivery adjuvants, such as ion exchange resins compatible with the ocular surface, which may also be present in the composition. In one embodiment, the composition provides a sustained concentration of the NSAID of between 10⁻⁸ and 10⁻⁴ M, in another embodiment between 10⁻⁷ and 10⁻⁵ M, in the aqueous or treated tissue of the eye for at least two hours, and in certain embodiments, at least three hours. In another embodiment, the composition of the disclosure provides sustained concentration of bromfenac of between 10⁻⁸ and 10⁻⁴ M, or between 10⁻⁷ and 10⁻⁵ M, in the aqueous or treated tissue of the eye for at least two hours, or at least three hours.

Ophthalmic compositions of the present disclosure may be formulated so that they retain the same or substantially the same viscosity in the eye that they had prior to administration to the eye. Alternatively, ophthalmic suspensions of the present disclosure may be formulated so that there is increased gelation upon contact with tear fluid. For instance, when a formulation containing DuraSite® at a pH of either below or above the pH of the eye which is about 7.2, is administered to the eye, the DuraSite® system swells upon contact with tears. This gelation or increase in gelation leads to a slower release rate of the therapeutic agent present in the composition, thereby extending the residence time and uptake of the therapeutic agent(s) in the eye. These events eventually lead to increased patient comfort, increase in the time the therapeutic agent(s) is/are in contact with the eye tissues, thereby increasing the extent of drug absorption and duration of action of the formulation in the eye and reducing the number of times the medicament must be applied to the eye to achieve a therapeutic effect.

Further provided is an ophthalmic vehicle with desirable rheological properties that are conducive to medicament delivery into the eye and provide corneal retention and in some such embodiments, comprise a modified Durasite® system. In some such embodiments, the vehicle uses a combination of an anionic carboxy-containing polymer in conjunction with a substantially smaller amount of a second polymer, for example, a cationic polymer such as chitosan. The second polymer is included at a sufficiently low concentration such that the particles of the carboxy-containing polymer remain suspended, yet when combined with the second polymer, the resulting vehicle has higher viscosity than the vehicle with the carboxy-containing polymer alone. The vehicle disclosed herein has the property that, when combined with tear fluid, its mucoadhesion increases due to the higher pH of tear fluid. The resultant viscosity provides a means by which to increase the efficiency of medicament delivery and corneal retention in the target tissue.

The ophthalmically acceptable vehicle disclosed herein also has suitable mucoadhesive properties that can facilitate the absorption of poorly absorbed drugs by increasing the contact time of the drug with the ocular mucosa. Interactions between the vehicle and the ocular mucosa can include Van der Waals attractive forces, hydrogen bonding, and electrostatic interactions between the mucins of the ocular mucosa and the carboxy-containing polymer and the second polymer. Together, these forces can increase the residence time of a medicament in the eye. An additional benefit of the ophthalmically acceptable vehicle disclosed herein, is the ability to provide the medicament in a sustained release manner.

In contrast to other systems, the present disclosure provides an ophthalmically acceptable vehicle that not only has the benefit of administration in drop form, but also does not suffer from breakdown limitations. Through administration at a viscosity such that the suspension can be reliably administered in drop form, but which actually increases when the suspension is so administered, controlled release of the active ingredient is significantly enhanced.

A viscosity substantially over 30,000 cps is not useful for drop formulations; when the viscosity is substantially lower than about 1,000 cps, the ability to gel upon contact with tears can be impeded and ocular retention is reduced. The increased gelation upon contact with the tears occurs with a pH change when a suspension having a pH of from about 3 to about 7.4 and an osmolality of from about 10 to about 400 mOsm/kg, contacts tear fluid, which has a higher pH of about 7.2 to about 8.0. Without being bound by the theory, with the pH increase, the carboxylic acid (COOH) functional group disassociates into carboxylate anions (COO⁻). Through electrostatic interactions, these carboxylate ions repel each other, causing the polymer to expand. The presence of the trace second polymer in the system can provide additional electrostatic, hydrogen bonding, and possible salt-bridge interactions with the mucins of the ocular mucosa, in addition to providing the initial beneficial viscosity modifying properties to the base vehicle. These chemical interactions result in enhanced controlled release of medicament from the vehicle.

The relationship of cross-linking and particle size can be significant. Because the particles are present in a suspension, the degree of cross-linking is necessarily at a level that avoids substantial dissolution of the polymer. On the other hand, since rapid gelation is achieved at the time of the pH change, the degree of cross-linking is necessarily not so great that gelation is precluded. Moreover, if the polymer particle size is too large, induced swelling can tend to take up voids in the volume between large particles that are in contact with one another, rather than the swelling tending to cause gelation.

In a suspension, particle size can be relevant to comfort. However, it has been found that in the system of the present disclosure, the small particle size and light cross-linking act synergistically to yield the observed rapid gelation when the pH is raised or lowered to the pH of the eye. Surprisingly, the use of particles greater than about 25 μm eliminates the observed gelation when the pH of the vehicle is increased. Moreover, at about the less than 25 μm size, there is also reasonably good eye comfort.

Exemplary commercially available lightly cross-linked carboxy-containing polymers useful in the present technology include, for example, polycarbophil (available, for example, from BF Goodrich, Cleveland, Ohio), a polyacrylic acid cross-linked with divinyl glycol. Without being bound by theory, this polymer benefits from its mucoadhesive properties which aid in increasing the residence time of the active ingredient in the eye. Other mucoadhesive polymers can be used in conjunction with, or in lieu of the lightly cross-linked polymers disclosed herein, for example, Carbopols such as 934P, 940, 941, 976, 971P, 974P, 980, 981 or hyaluronic acid. The latter has been demonstrated to be an effective mucoadhesive polymer in ocular formulations (Saettone et al. Int. J. Pharm. 51: 203-212, (1989)).

Aqueous suspensions containing polymer particles prepared by suspension or emulsion polymerization whose average dry particle size is appreciably larger than about 10 μm in equivalent spherical diameter are less comfortable when administered to the eye than suspensions otherwise identical in composition containing polymer particles whose equivalent spherical diameters are, on the average, below about 10 μm. Moreover, above the average 5.0 μm size, the advantage of substantially increased viscosity after administration is not realized. It has also been discovered that lightly cross-linked polymers of acrylic acid or the like prepared to a dry particle size appreciably larger than about 5.0 μm in equivalent spherical diameter and then reduced in size, e.g., by mechanically milling or grinding, to a dry particle size of not more than about 5.0 μm in equivalent spherical diameter do not work as well as polymers made from aqueous suspensions in the ophthalmic vehicle of the present technology.

While not being bound by any theory or mechanism, one possible explanation for the difference of such mechanically milled or ground polymer particles as the sole particulate polymer present is that grinding disrupts the spatial geometry or configuration of the larger than 5.0 μm lightly cross-linked polymer particles, perhaps by removing uncross-linked branches from polymer chains, by producing particles having sharp edges or protrusions, or by producing ordinarily too broad a range of particle sizes to afford satisfactory delivery system performance. A broad distribution of particle sizes impairs the viscosity-gelation relationship. In any event, such mechanically reduced particles are less easily hydratable in aqueous suspension than particles prepared to the appropriate size by suspension or emulsion polymerization, and also are less able to gel in the eye under the influence of tear fluid to a sufficient extent and are less comfortable once gelled than gels produced in the eye using the aqueous suspensions of this disclosure. However, up to about, 40% by weight, e.g., from about 0% to over 20% by weight, based on the total weight of lightly cross-linked particles present, of such milled or ground polymer particles can be admixed with solution or emulsion polymerized polymer particles having dry particle diameters of not more than about 50 μm when practicing this inventive concept. Such mixtures also provide satisfactory viscosity levels in the ophthalmically acceptable vehicle and in the in situ gels formed in the eye coupled with ease and comfort of administration and satisfactory sustained release of the active ingredient to the eye, particularly when such milled or ground polymer particles, in dry form, average from about 0.01 to about 30 μm, and in other embodiments, from about 1 to about 5 μm, in equivalent spherical diameter.

In the ophthalmically acceptable vehicle, chitosan or other second polymer is present in an amount ranging from about 0.01% to about 1.0% when using a cationic polymer having a molecular weight ranging from about 500 kDa to about 3000 kDa. The amount of cationic polymer or chitosan can be any amount in between, including about 0.01%, 0.025%, 0.05%. 0.075%, 0.10%, 0.15%, 0.20%, 0.25%, 0.30%, 0.35%, 0.40%, 0.45%, 0.5%, 0.75% and 1.0% and any amount in between these values. When using higher molecular weight cationic polymers, such as in a range from about 1,000 to about 3,000 kDa, the amount of cationic polymer necessary to achieve favorable viscosities can be substantially reduced. For example, the amount of 1,000 kDa to about 3,000 kDa chitosan can be in a range in a range from about 0.01% and 0.5%, or any amount in between including, for example, 0.01%, 0.015%, 0.020%, 0.025%, 0.030%, 0.035%, 0.040%, 0.045%, 0.05%, 0.1%, 0.15%, 0.20%, 0.25%, 0.30%, 0.35%, 0.40%, 0.45%, 0.50%, −/75% and 1.0%.

When formulating the aqueous suspensions, their osmolality will be adjusted to from about 10 mOsm/kg to about 400 mOsm/kg, and in other embodiments, from about 100 to about 300 mOsm/kg, using appropriate amounts of physiologically and ophthalmologically acceptable salts. Sodium chloride can be used as an osmolality adjusting agent to adjust the osmolality of the aqueous suspension to approximate that of physiologic fluid. The amounts of sodium chloride ranging from about 0.01% to about 1% by weight, and in other embodiments from about 0.05% to about 0.45% by weight, based on the total weight of the aqueous suspension, will give osmolalities within the above-stated ranges. Equivalent amounts of one or more salts made up of cations such as potassium, ammonium and the like and anions such as chloride, citrate, ascorbate, borate, phosphate, bicarbonate, sulfate, thiosulfate, bisulfite and the like, e.g., potassium chloride, sodium thiosulfate, sodium bisulfite, ammonium sulfate, and the like can also be used in addition to or instead of sodium chloride to achieve osmolalities within the above-stated ranges.

The active ingredient-ophthalmically acceptable vehicle can be formulated in any of several ways. For example the active ingredient, lightly cross-linked polymer particles, and osmolality-adjusting agent can be pre-blended in dry form, added to all or part of the water, and stirred vigorously until apparent polymer dispersion is complete, as evidenced by the absence of visible polymer aggregates. Sufficient pH adjusting agent is then added incrementally to reach the desired pH, and more water to reach 100 percent formula weight can be added at this time, if necessary. Another convenient method involves adding the drug to about 95 percent of the final water volume and stirring for a sufficient time to saturate the solution. Solution saturation can be determined in any known manner, e.g., using a spectrophotometer. The lightly cross-linked polymer particles and the osmolality-adjusting agent are first blended in dry form and then added to the drug-saturated suspension and stirred until apparent polymer hydration is complete. Following the incremental addition of sufficient pH adjusting agent to reach the desired pH, the remainder of the water is added, with stirring, to bring the suspension to 100 percent formula weight.

These aqueous suspensions can be packaged in preservative-free, single-dose non-reclosable containers. This permits a single dose of the active ingredient to be delivered to the eye one drop at a time, with the container then being discarded after use. Such containers eliminate the potential for preservative-related irritation and sensitization of the corneal epithelium, as has been observed to occur particularly from ophthalmic medicaments containing mercurial preservatives. Multiple-dose containers can also be used, if desired, particularly since the relatively low viscosities of the aqueous suspensions of this invention permit constant, accurate dosages to be administered dropwise to the eye as many times each day as necessary.

In those vehicles where preservatives are to be included, suitable preservatives are chlorobutanol, Polyquat, benzalkonium chloride, cetyl bromide, benzethonium chloride, cetyl pyridinium chloride, benzyl bromide, EDTA, phenylmercury nitrate, phenylmercury acetate, thimerosal, merthiolate, acetate and phenylmercury borate, chlorhexidine, polymyxin B sulphate, methyl and propyl parabens, phenylethyl alcohol, quaternary ammonium chloride, sodium benzoate, sodium proprionate, sorbic acid, and sodium perborate. In particular embodiments, the preservative includes benzalkonium chloride.

The composition containing a medicament and an ophthalmically acceptable vehicle can be individually packaged for a single dose administration, e.g., in a bottle, jar, ampoule, tube, syringe, envelope, container, unit dose container or vial. When the composition is individually packaged, in some embodiments, the composition does not include a preservative. Alternatively, the composition can be contained in a package that is capable of holding multiple units, e.g., in resealable glass or plastic eyedropper bottles.

In an embodiment, according to any of the above aspects, provided is a composition or method for combination therapy of the eye and/or surrounding tissue of a mammal including: an ophthalmic composition having a therapeutically effective amount of an NSAID, a glucocorticoid, and a flowable mucoadhesive polymer such as DuraSite® or modified DuraSite® (e.g., DuraSite® plus chitosan) as described above, wherein the composition has a viscosity formulated for topical administration to the eye of a mammal in drop form. In another embodiment, provided is a composition or method for combination therapy of the eye of a mammal including: an topical administration of an ophthalmic composition having a therapeutically effective amount of an NSAID and a glucocorticoid, in an ophthalmic vehicle comprising a flowable mucoadhesive polymer such as DuraSite® or modified DuraSite® and one or more different additional NSAIDs. In another embodiment, there is provided a composition or method for combination therapy of the eye of a mammal including: topical administration of an ophthalmic composition having a therapeutically effective amount of an NSAID, a glucocorticoid, and a flowable mucoadhesive polymer such as DuraSite® or modified DuraSite® and one or more additional steroidal anti-inflammatory agents. In another embodiment, provided is a composition or method for combination therapy of the eye of a mammal including: topical administration of an ophthalmic composition having a therapeutically effective amount of an NSAID and a glucocorticoid, a flowable mucoadhesive polymer such as DuraSite® or modified DuraSite® and one or more antibacterial agent. In another embodiment, relating to any of the above aspects, the inventive disclosure relates to a composition or method for combination therapy of the eye of a mammal including: an ophthalmic composition having a therapeutically effective amount of bromfenac and dexamethasone, a flowable mucoadhesive polymer such as DuraSite® or modified DuraSite® and an additional therapeutically active agent selected from the group consisting of antibiotic agent, synthetic antibacterial agent, antifungal antibiotic agent, synthetic antifungal agent, antineoplastic agent, a second steroidal anti-inflammatory agent, a second non-steroidal anti-inflammatory agent, anti-allergic agent, glaucoma-treating agent, antiviral agent and anti-mycotic agent.

In some embodiments, according to any of the above embodiments the compositions can include, in addition to the a first NSAID and glucocorticoid, one or more other active ingredients such as other NSAIDs. Suitable additional NSAIDs for combination therapy are, for example, aspirin, benoxaprofen, benzofenac, bucloxic acid, butibufen, carprofen, cicloprofen, cinmetacin, clidanac, clopirac, diclofenac, diflupredinate, etodolac, fenbufen, fenclofenac, fenclorac, fenoprofen, fentiazac, flunoxaprofen, furaprofen, flurbiprofen, furobufen, furofenac, ibuprofen, ibufenac, indomethacin, indoprofen, isoxepac, ketorolac, ketroprofen, lactorolac, lonazolac, metiazinic, miroprofen, nepafenac, naproxen, norketotifen, oxaprozin, oxepinac, phenacetin, pirprofen, pirazolac, protizinic acid, sulindac, suprofen, tiaprofenic acid, tolmetin, and zomepirac.

Unless the intended purpose of use is affected adversely, the ophthalmic formulation of the present invention can further comprise one or more additional therapeutically-active agents. Specific therapeutically-active agents include, but are not limited to: antibacterial antibiotics, synthetic antibacterials, antifungal antibiotics, synthetic antifungals, antineoplastic agents, further steroidal anti-inflammatory agents, further non-steroidal anti-inflammatory agents, anti-allergic agents, glaucoma-treating agents, antiviral agents, and anti-mycotic agents. Further contemplated are any derivatives of the therapeutically-active agents which may include, but not be limited to: analogs, salts, esters, amines, amides, alcohols and acids derived from an agent of the invention and may be used in place of an agent itself.

Examples of the antibiotics include, but are not limited to: aminoglycosides (e.g., amikacin, apramycin, arbekacin, bambermycins, butirosin, dibekacin, dihydrostreptomycin, fortimicin(s), gentamicin, isepamicin, kanamycin, micronomicin, neomycin, neomycin undecylenate, netilmicin, paromomycin, ribostamycin, sisomicin, spectinomycin, streptomycin, tobramycin, trospectomycin), amphenicols (e.g., azidamfenicol, chloramphenicol, florfenicol, thiamphenicol), ansamycins (e.g., rifamide, rifampin, rifamycin sv, rifapentine, rifaximin), beta-lactams (e.g., carbacephems (e.g., loracarbef), carbapenems (e.g., biapenem, imipenem, meropenem, panipenem), cephalosporins (e.g., cefaclor, cefadroxil, cefamandole, cefatrizine, cefazedone, cefazolin, cefcapene pivoxil, cefclidin, cefdinir, cefditoren, cefepime, cefetamet, cefixime, cefmenoxime, cefodizime, cefonicid, cefoperazone, ceforanide, cefotaxime, cefotiam, cefozopran, cefpimizole, cefpiramide, cefpirome, cefpodoxime proxetil, cefprozil, cefroxadine, cefsulodin, ceftazidime, cefteram, ceftezole, ceftibuten, ceftizoxime, ceftriaxone, cefuroxime, cefuzonam, cephacetrile sodium, cephalexin, cephaloglycin, cephaloridine, cephalosporin, cephalothin, cephapirin sodium, cephradine, pivcefalexin), cephamycins (e.g., cefbuperazone, cefmetazole, cefininox, cefotetan, cefoxitin), monobactams (e.g., aztreonam, carumonam, tigemonam), oxacephems, flomoxef, moxalactam), penicillins (e.g., amdinocillin, amdinocillin pivoxil, amoxicillin, ampicillin, apalcillin, aspoxicillin, azidocillin, azlocillin, bacampicillin, benzylpenicillinic acid, benzylpenicillin sodium, carbenicillin, carindacillin, clometocillin, cloxacillin, cyclacillin, dicloxacillin, epicillin, fenbenicillin, floxacillin, hetacillin, lenampicillin, metampicillin, methicillin sodium, mezlocillin, nafcillin sodium, oxacillin, penamecillin, penethamate hydriodide, penicillin g benethamine, penicillin g benzathine, penicillin g benzhydrylamine, penicillin g calcium, penicillin g hydrabamine, penicillin g potassium, penicillin g procaine, penicillin n, penicillin o, penicillin v, penicillin v benzathine, penicillin v hydrabamine, penimepicycline, phenethicillin potassium, piperacillin, pivampicillin, propicillin, quinacillin, sulbenicillin, sultamicillin, talampicillin, temocillin, ticarcillin), other (e.g., ritipenem), lincosamides (e.g., clindamycin, lincomycin), macrolides (e.g., azithromycin, carbomycin, clarithromycin, dirithromycin, erythromycin, erythromycin acistrate, erythromycin estolate, erythromycin glucoheptonate, erythromycin lactobionate, erythromycin propionate, erythromycin stearate, josamycin, leucomycins, midecamycins, miokamycin, oleandomycin, primycin, rokitamycin, rosaramicin, roxithromycin, spiramycin, troleandomycin), polypeptides (e.g., amphomycin, bacitracin, capreomycin, colistin, enduracidin, enviomycin, fusafungine, gramicidin s, gramicidin(s), mikamycin, polymyxin, pristinamycin, ristocetin, teicoplanin, thiostrepton, tuberactinomycin, tyrocidine, tyrothricin, vancomycin, viomycin, virginiamycin, zinc bacitracin), tetracyclines (e.g., apicycline, chlortetracycline, clomocycline, demeclocycline, doxycycline, guamecycline, lymecycline, meclocycline, methacycline, minocycline, oxytetracycline, penimepicycline, pipacycline, rolitetracycline, sancycline, tetracycline), and others (e.g., cycloserine, mupirocin, tuberin).

Examples of the synthetic antibacterials include, but are not limited to: 2,4-diaminopyrimidines (e.g., brodimoprim, tetroxoprim, trimethoprim), nitrofurans (e.g., furaltadone, furazolium chloride, nifuradene, nifuratel, nifurfoline, nifurpirinol, nifurprazine, nifurtoinol, nitrofurantoin), quinolones and analogs (e.g., cinoxacin, ciprofloxacin, clinafloxacin, difloxacin, enoxacin, fleroxacin, flumequine, grepafloxacin, lomefloxacin, miloxacin, nadifloxacin, nalidixic acid, norfloxacin, ofloxacin, oxolinic acid, pazufloxacin, pefloxacin, pipemidic acid, piromidic acid, rosoxacin, rufloxacin, sparfloxacin, temafloxacin, tosufloxacin, trovafloxacin), sulfonamides (e.g., acetyl sulfamethoxypyrazine, benzylsulfamide, chloramine-b, chloramine-t, dichloramine-t, n.sup.2-formylsulfisomidine, n.sup.4-beta-d-glucosyl sulfanilamide, mafenide, 4′-(methylsulfamoyl)sulfanilanilide, noprylsulfamide, phthalylsulfacetamide, phthalylsulfathiazole, salazosulfadimidine, succinylsulfathiazole, sulfabenzamide, sulfacetamide, sulfachlorpyridazine, sulfachrysoidine, sulfacytine, sulfadiazine, sulfadicramide, sulfadimethoxine, sulfadoxine, sulfaethidole, sulfaguanidine, sulfaguanol, sulfalene, sulfaloxic acid, sulfamerazine, sulfameter, sulfamethazine, sulfamethizole, sulfamethomidine, sulfamethoxazole, sulfamethoxypyridazine, sulfametrole, sulfamidocchrysoidine, sulfamoxole, sulfanilamide, 4-sulfanilamidosalicylic acid, n.sup.4-sulfanilylsulfanilamide, sulfanilylurea, n-sulfanilyl-3,4-xylamide, sulfanitran, sulfaperine, sulfaphenazole, sulfaproxyline, sulfapyrazine, sulfapyridine, sulfasomizole, sulfasymazine, sulfathiazole, sulfathiourea, sulfatolamide, sulfisomidine, sulfisoxazole) sulfones (e.g., acedapsone, acediasulfone, acetosulfone sodium, dapsone, diathymosulfone, glucosulfone sodium, solasulfone, succisulfone, sulfanilic acid, p-sulfanilylbenzylamine, sulfoxone sodium, thiazolsulfone), and others (e.g., clofoctol, hexedine, methenamine, methenamine anhydromethylene-citrate, methenamine hippurate, methenamine mandelate, methenamine sulfosalicylate, nitroxoline, taurolidine, xibornol).

Examples of further steroidal anti-inflammatory agents include, but are not limited to: 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, and triamcinolone hexacetonide.

Examples of the antifungal antibiotics include, but are not limited to: polyenes (e.g., amphotericin b, candicidin, dennostatin, filipin, fungichromin, hachimycin, hamycin, lucensomycin, mepartricin, natamycin, nystatin, pecilocin, perimycin), others (e.g., azaserine, griseofulvin, oligomycins, neomycin undecylenate, pyrroInitrin, siccanin, tubercidin, viridin). Examples of the synthetic antifungals include, but are not limited to: allylamines (e.g., butenafine, naftifine, terbinafine), imidazoles (e.g., bifonazole, butoconazole, chlordantoin, chlormiidazole, clotrimazole, econazole, enilconazole, fenticonazole, flutrimazole, isoconazole, ketoconazole, lanoconazole, miconazole, omoconazole, oxiconazole nitrate, sertaconazole, sulconazole, tioconazole), thiocarbamates (e.g., tolciclate, tolindate, tolnaftate), triazoles (e.g., fluconazole, itraconazole, saperconazole, terconazole) others (e.g., acrisorcin, amorolfine, biphenamine, bromosalicylchloranilide, buclosamide, calcium propionate, chlorphenesin, ciclopirox, cloxyquin, coparaffinate, diamthazole dihydrochloride, exalamide, flucytosine, halethazole, hexetidine, loflucarban, nifuratel, potassium iodide, propionic acid, pyrithione, salicylanilide, sodium propionate, sulbentine, tenonitrozole, triacetin, ujothion, undecylenic acid, zinc propionate).

In general, ophthalmic formulations suitable for topical ophthalmic administration can be formulated and administered in accordance with techniques familiar to persons skilled in the art. The finished formulations are stored in opaque or brown containers to protect them from light exposure, and under an inert atmosphere. These compositions can be packaged in preservative-free, single-dose non-reclosable/reclosable containers or kits. This permits a single dose of the medicament to be delivered to the eye as a drop, with the container then being discarded after use. Such containers eliminate the potential for preservative-related irritation and sensitization of the corneal epithelium, as has been observed to occur particularly from ophthalmic medicaments containing mercurial preservatives. Multiple dose containers can also be used, if desired, particularly since the relatively low viscosities of the compositions of this invention permit constant, accurate dosages to be administered dropwise to the eye as many times each day as necessary. In those suspensions where preservatives are to be included, suitable preservatives are chlorobutanol, polyquat, benzalkonium chloride, cetyl bromide, sorbic acid and the like.

An additional embodiment includes the method of treating ocular pain and/or inflammation in a patient in need thereof, wherein said inflammation and accompanying pain is the result of allergic, viral or bacterial conjunctivitis, and wherein said treatment comprises treating the patient with any of the disclosed formulations.

An additional embodiment includes a method of treating ocular pain and/or inflammation associated with allergic, viral or bacterial conjunctivitis with one of the topical ophthalmic formulations of the invention. An additional embodiment may include one or more additional active ingredients as part of the formulation. Such additional actives may include, but are not limited to, antihistamines and/or antibacterials and/or antimicrobial compounds, to further assist with the treatment of the conjunctivitis condition.

An additional embodiment includes a method for treating an eye wherein its normal condition has been disrupted or changed comprising administering to said eye one to six times daily a formulation or composition of the invention. An additional embodiment of includes a method for treating postoperative inflammation and/or pain in patients who have undergone cataract extraction comprising the once, twice or up to six times daily administration of a selected formulation into the effected eye.

For example, an embodiment provides a process for therapeutic treatment of an inflammatory condition of the eye in a mammal including: (a) providing an ophthalmic composition comprising an NSAID and glucocorticoid, each in an amount of about 0.005% to about 0.5% by weight of the composition and a flowable mucoadhesive polymer in an amount of about 0.5% to about 1.5% by weight of the composition; (b) administering said composition to the eye of a mammal in need thereof to treat inflammation or inflammatory conditions of the eye. In a related embodiment, the ophthalmic composition further includes a therapeutically active agent selected from the group consisting of antibacterial antibiotic agent, synthetic antibacterial agent, antifungal antibiotic, synthetic antifungal agent, antineoplastic agent, steroidal anti-inflammatory agent, non-steroidal anti-inflammatory agent, anti-allergic agent, glaucoma-treating agent, antiviral agent and anti-mycotic agent.

The inflammatory conditions for which the compositions and methods can be used include, but are not limited to, surgical trauma; dry eye; allergic conjunctivitis; viral conjunctivitis; bacterial conjunctivitis; blepharitis; anterior uveitis; injury from a chemical; radiation or thermal burn; injury from penetration of a foreign body, pain in or around the eye, redness especially accompanied by pain in the eye; light sensitivity; seeing halos (colored circles or halos around lights); bulging (protrusion) of the eye; swelling of eye tissues; discharge, crusting or excessive tearing; eyelids stuck together, blood inside the front of the eye (on the colored part) or white of the eye; cataracts; pain and inflammation associated with wearing contact lenses; corneal-associated condition; conjunctival tumor excision; conjunctivitis known as Pink Eye; cornea edema after cataract surgery; corneal clouding; corneal transplantation; corneal ulcer; dry eye syndrome; dystrophies; condition associated with excimer laser phototherapeutic keratectomy; herpes simplex keratitis; keratoconus; pterygium; recurrent erosion syndrome; eye movement disorder; glaucoma; ocular oncology; oculoplastic condition resulted from cosmetic surgery, enucleation, eyelid and orbit injuries, ectropion, entropion, Graves' disease, involuntary eyelid blinking; condition associated with refractive surgery; and retinal condition.

The retinal conditions for which the compositions and methods can be used include but are not limited to, macular degeneration, AIDS-related ocular disease, CMV retinitis, birdshot retinochoroidopathy (BR), choroidal melanoma, coats disease, cotton wool spots, diabetic retinopathy diabetic macular edema, cystoid macular edema, lattice degeneration, macular disease, macular degeneration, hereditary macular dystrophy, macular edema, macular hole, macular pucker, central serous chorioretinopathy, ocular histoplasmosis syndrome (OHS), posterior vitreous detachment, retinal detachment, retinal artery obstruction, retinal vein occlusion, retinoblastoma, retinopathy of prematurity (ROP), retinitis pigmentosa, retinoschisis (acquired and x-linked), stargardt's disease, toxoplasmosis of retina or uveitis. The ophthalmic composition of bromfenac/dexamethasone is particularly effective in the treatment of cystoid macular edema.

In order that those skilled in the art can more fully appreciate aspects of this disclosure, the following Tables and examples are set forth. These examples are given solely for purposes of illustration and should not be considered as expressing limitations.

### Example I

This Example shows the preparation of exemplary formulations, in accordance with some embodiments of the present disclosure.

TABLE 1

| Component | 1 | 2 | 3 | 4 | 5 | 6 | 7 | 8 | 9 | 10 |
|---|---|---|---|---|---|---|---|---|---|---|
| Polycarbophil | 0.9 | 0.9 | 0.9 | 0.9 | 0.9 | 0.9 | 0.95 | 0.95 | 0.85 | 0.85 |
| Bromfenac | 0.09 | 0.075 | 0.01 | 0.04 | 0.075 | 0.075 | 0.075 | 0.075 | 0.075 | 0.075 |
| Dexamethasone | 0.1 | 0.1 | 0.05 | 0.05 | 0.05 | 0.1 | 0.05 | 0.1 | 0.1 | 0.1 |
| Poloxamer 407 | 0.2 | 0.2 | 0.2 | 0.2 | 0.2 | 0.2 | 0.2 | 0.2 | 0.2 | 0.2 |
| Sodium Edetate | 0.1 | 0.1 | 0.1 | 0.1 | 0.1 | 0.1 | 0.1 | 0.1 | | |
| Chitosan | | | | | | | 0.025 | 0.025 | 0.025 | 0.025 |
| Hydrochloric | | | | | | | 2.65 | 2.65 | 2.65 | 2.65 |
| Acid 2N | | | | | | | | | | |
| Tromethamine | 0.85 | 0.85 | 0.85 | 0.85 | 0.85 | 0.85 | 0.85 | 0.85 | 0.8 | 0.8 |
| Sodium Citrate | 0.2 | 0.2 | 0.2 | 0.2 | 0.2 | 0.2 | | | — | — |
| Citric Acid | 0.14 | 0.14 | 0.14 | 0.14 | 0.14 | 0.14 | | | — | — |
| Sodium Chloride | 0.1 | 0.1 | 0.1 | 0.1 | 0.1 | 0.1 | 0.025 | 0.025 | 0.35 | 0.35 |
| Mannitol | — | — | — | — | — | — | 0.4 | 0.4 | — | — |
| Benzalkonium | 0.003 | 0.003 | 0.003 | 0.003 | 0.003 | — | 0.003 | 0.003 | 0.005 | — |
| chloride | | | | | | | | | | |
| Sodium Hydroxide | qs to | qs to | qs to 8.3 | qs to 8.3 | qs to 8.3 | qs to 8.3 | qs to 8.3 | qs to 8.3 | qs to 8.5-8.6 | qs to 8.5-8.6 |
| | 8.3 | 8.3 | | | | | | | | |
| Water, USP | qs to | qs to | qs to | qs to | qs to | qs to | qs to | qs to | qs to | qs to |
| | 100% | 100% | 100% | 100% | 100% | 100% | 100% | 100% | 100% | 100% |

TABLE 2

| Component | 1 | 3 | 3 | 4 | 5 | 6 | 7 | 8 |
|---|---|---|---|---|---|---|---|---|
| Polycarbophil | 0.9 | 0.9 | 0.95 | 0.95 | 0.9 | 0.9 | 0.95 | 0.95 |
| Ketorolac | 0.2 | 0.4 | 0.2 | 0.4 | 0.2 | 0.4 | 0.2 | 0.4 |
| tromethamine | | | | | | | | |
| Dexamethasone | 0.1 | 0.1 | 0.1 | 0.1 | 0.1 | 0.1 | 0.1 | 0.1 |
| Hydrochloric | | | 1.5 | 1.5 | | | 1.5 | 1.5 |
| Acid 2N | | | | | | | | |
| Chitosan | | | 0.025 | 0.025 | | | 0.025 | 0.025 |
| Octoxynol 40 | 0.05 | 0.05 | 0.05 | 0.05 | 0.05 | 0.05 | 0.05 | 0.05 |
| Sodium Edetate | 0.1 | 0.1 | 0.1 | 0.1 | 0.1 | 0.1 | 0.1 | 0.1 |
| Sodium Citrate | 0.2 | 0.2 | | | 0.2 | 0.2 | | |
| Citric Acid | 0.14 | 0.14 | | | 0.14 | 0.14 | | |
| Sodium Chloride | 0.45 | 0.45 | 0.35 | 0.35 | 0.45 | 0.45 | 0.35 | 0.35 |
| Mannitol | 1.0 | 1.0 | 1.0 | 1.0 | 1.0 | 1.0 | 1.0 | 1.0 |
| Benzalkonium | 0.005 | 0.005 | 0.005 | 0.005 | | | — | — |
| chloride | | | | | | | | |
| Sodium | qs to 6.3 | qs to 6.3 | qs to 6.3 | qs to 6.3 | qs to 6.3 | qs to 6.3 | qs to 6.3 | qs to 6.3 |
| Hydroxide | | | | | | | | |
| Water, USP | qs to 100% | qs to 100% | qs to 100% | qs to 100% | qs to 100% | qs to 100% | qs to 100% | qs to 100% |

TABLE 3

| Component | 1 | 3 | 3 | 4 | 5 | 6 | 7 | 8 |
|---|---|---|---|---|---|---|---|---|
| Polycarbophil | 0.9 | 0.9 | 0.95 | 0.95 | 0.9 | 0.9 | 0.95 | 0.95 |
| Ketorolac | 0.2 | 0.4 | 0.2 | 0.4 | 0.2 | 0.4 | 0.2 | 0.4 |
| tromethamine | | | | | | | | |
| prednisolone | 1.0 | 1.0 | 1.0 | 1.0 | 1.0 | 1.0 | 1.0 | 1.0 |
| acetate | | | | | | | | |
| Hydrochloric | | | 1.5 | 1.5 | | | 1.5 | 1.5 |
| Acid 2N | | | | | | | | |
| Chitosan | | | 0.025 | 0.025 | | | 0.025 | 0.025 |
| Octoxynol 70 | 0.05 | 0.05 | 0.05 | 0.05 | 0.05 | 0.05 | 0.05 | 0.05 |
| Sodium Edetate | 0.1 | 0.1 | 0.1 | 0.1 | 0.1 | 0.1 | 0.1 | 0.1 |
| Sodium Citrate | 0.2 | 0.2 | | | 0.2 | 0.2 | | |
| Citric Acid | 0.14 | 0.14 | | | 0.14 | 0.14 | | |
| Sodium Chloride | 0.45 | 0.45 | 0.35 | 0.35 | 0.45 | 0.45 | 0.35 | 0.35 |
| Mannitol | 1.0 | 1.0 | 1.0 | 1.0 | 1.0 | 1.0 | 1.0 | 1.0 |
| Benzalkonium | 0.003 | 0.003 | 0.003 | 0.003 | 0.003 | 0.003 | — | — |
| chloride | | | | | | | | |
| Sodium | qs to 6.3 | qs to 6.3 | qs to 6.3 | qs to 6.3 | qs to 6.3 | qs to 6.3 | qs to 6.3 | qs to 6.3 |
| Hydroxide | | | | | | | | |
| Water, USP | qs to 100% | qs to 100% | qs to 100% | qs to 100% | qs to 100% | qs to 100% | qs to 100% | qs to 100% |

TABLE 4

| Component | 1 | 3 | 3 | 4 | 5 | 6 | 7 | 8 |
|---|---|---|---|---|---|---|---|---|
| Polycarbophil | 0.9 | 0.9 | 0.95 | 0.95 | 0.9 | 0.9 | 0.95 | 0.95 |
| nepafenac | 0.1 | 0.3 | 0.1 | 0.3 | 0.1 | 0.3 | 0.1 | 0.3 |
| dexamethasone | 1.0 | 1.0 | 1.0 | 1.0 | 1.0 | 1.0 | 1.0 | 1.0 |
| Poloxamer 407 | 0.2 | 0.2 | 0.2 | 0.2 | 0.2 | 0.2 | 0.2 | 0.2 |
| Hydrochloric | | | 1.5 | 1.5 | | | 1.5 | 1.5 |
| Acid 2N | | | | | | | | |
| Chitosan | | | 0.025 | 0.025 | | | 0.025 | 0.025 |
| Sodium Edetate | 0.1 | 0.1 | 0.1 | 0.1 | 0.1 | 0.1 | 0.1 | 0.1 |
| Sodium Citrate | 0.2 | 0.2 | | | 0.2 | 0.2 | | |
| Citric Acid | 0.14 | 0.14 | | | 0.14 | 0.14 | | |
| Sodium Chloride | 0.45 | 0.45 | 0.35 | 0.35 | 0.45 | 0.45 | 0.35 | 0.35 |
| Mannitol | 1.0 | 1.0 | 1.0 | 1.0 | 1.0 | 1.0 | 1.0 | 1.0 |
| Benzalkonium | 0.006 | 0.006 | 0.006 | 0.006 | | | — | — |
| chloride | | | | | | | | |
| Sodium | qs to 6.8 | qs to 6.8 | qs to 6.3 | qs to 6.8 | qs to 6.8 | qs to 6.8 | qs to | qs to 6.8 |
| Hydroxide | | | | | | | 6.8 | |
| Water, USP | qs to 100% | qs to 100% | qs to 100% | qs to 100% | qs to 100% | qs to 100% | qs to 100% | qs to 100% |

TABLE 5

| Component | 1 | 3 | 3 | 4 | 5 | 6 | 7 | 8 |
|---|---|---|---|---|---|---|---|---|
| Polycarbophil | 0.9 | 0.9 | 0.9 | 0.9 | 0.9 | 0.9 | 0.9 | 0.9 |
| nepafenac | 0.1 | 0.3 | 0.1 | 0.3 | 0.1 | 0.3 | 0.1 | 0.3 |
| prednisolone | 1.0 | 1.0 | 1.0 | 1.0 | 1.0 | 1.0 | 1.0 | 1.0 |
| Hydrochloric | | | 1.5 | 1.5 | | | 1.5 | 1.5 |
| Acid 2N | | | | | | | | |
| Chitosan | | | 0.025 | 0.025 | | | 0.025 | 0.025 |
| Poloxamer 407 | 0.2 | 0.2 | 0.2 | 0.2 | 0.2 | 0.2 | 0.2 | 0.2 |
| Sodium Edetate | 0.1 | 0.1 | 0.1 | 0.1 | 0.1 | 0.1 | 0.1 | 0.1 |
| Sodium Citrate | 0.2 | 0.2 | | | 0.2 | 0.2 | | |
| Citric Acid | 0.14 | 0.14 | | | 0.14 | 0.14 | | |
| Sodium Chloride | 0.45 | 0.45 | 0.35 | 0.35 | 0.45 | 0.45 | 0.35 | 035 |
| Mannitol | 1.0 | 1.0 | 1.0 | 1.0 | 1.0 | 1.0 | 1.0 | 1.0 |
| Benzalkonium | 0.003 | 0.003 | 0.003 | 0.003 | | | — | — |
| chloride | | | | | | | | |
| Sodium | qs to 6.3 | qs to 6.3 | qs to 6.8 | qs to 6.8 | qs to 6.8 | qs to 6.8 | qs to 6.8 | qs to 6.8 |
| Hydroxide | | | | | | | | |
| Water, USP | qs to 100% | qs to 100% | qs to 100% | qs to 100% | qs to 100% | qs to 100% | qs to 100% | qs to 100% |

TABLE 6

| Component | 1 | 3 | 3 | 4 | 5 | 6 | 7 | 8 |
|---|---|---|---|---|---|---|---|---|
| Polycarbophil | 0.9 | 0.9 | 0.95 | 0.95 | 0.925 | 0.925 | 0.95 | 0.95 |
| Ketorolac | — | — | — | — | — | — | 0.4 | 0.2 |
| tromethamine | | | | | | | | |
| Nepafenac | 0.1 | 0.3 | 0.1 | 0.3 | 0.1 | 0.3 | — | — |
| Fluoromethalone | 0.1 | 0.1 | — | — | — | — | — | — |
| Loteprednol | — | — | 0.5 | 0.5 | — | — | 0.5 | 0.5 |
| Etabonate | | | | | | | | |
| Difluprednate | — | — | — | — | 0.05 | 0.05 | — | — |
| Hydrochloric | — | — | 1.5 | 1.5 | — | — | 1.5 | 1.5 |
| Acid 2N | | | | | | | | |
| Chitosan | — | — | 0.025 | 0.025 | — | — | 0.025 | 0.025 |
| Poloxamer 407 | 0.2 | 0.2 | 0.2 | 0.2 | 0.5 | 0.5 | 0.2 | 0.2 |
| Mineral oil | — | — | — | — | 10 | 10 | — | — |
| Sodium Edetate | 0.1 | 0.1 | 0.1 | 0.1 | 0.1 | 0.1 | 0.1 | 0.1 |
| Sodium Citrate | 0.2 | 0.2 | — | — | 0.2 | 0.2 | — | — |
| Citric Acid | 0.14 | 0.14 | — | — | 0.14 | 0.14 | — | — |
| Sodium Chloride | 0.45 | 0.45 | 0.35 | 0.35 | 0.25 | 0.25 | 0.35 | 035 |
| Mannitol | 1.0 | 1.0 | 1.0 | 1.0 | 1.0 | 1.0 | 1.0 | 1.0 |
| Benzalkonium | 0.003 | 0.003 | 0.003 | 0.003 | 0.003 | 0.003 | 0.003 | 0.003 |
| chloride | | | | | | | | |
| Sodium | qs to 6.8 | qs to 6.8 | qs to 6.8 | qs to 6.8 | qs to 6.8 | qs to 6.8 | qs to 6.8 | qs to 6.8 |
| Hydroxide | | | | | | | | |
| Water, USP | qs to 100% | qs to 100% | qs to 100% | qs to 100% | qs to 100% | qs to 100% | qs to 100% | qs to 100% |

Formulations 1-10 in Table 1 and Formulations 1-8 in Tables 2-6 are made by adding polycarbophil, sodium chloride and edetate to water by stirring for 0.5 hours. The solution is then sterilized at 121° C. for 45 minutes and cooled to room temperature. The citrate buffer is dissolved in water and added by sterile addition through a 0.2 um filter while mixing. The mannitol, poloxamer, and NSAID are dissolved in water and added to the batch by sterile addition. The steroidal anti-inflammatory which has been sterilized by Co-60 radiation is added to the batch by sterile dry particle addition and mixed into the batch. The tromethamine buffer and benzalkonium chloride are dissolved and added by sterile filtration while mixing. Sodium hydroxide is added by sterile addition to adjust the pH to the target value.

For formulations that include chitosan, an aqueous solution of chitosan is prepared using hydrochloric acid and the solution is sterile filtered into the sterilized polycarbophil suspension.

The embodiments within the specification provide an illustration of embodiments and should not be construed to limit the scope of the invention. The skilled artisan readily recognizes that many other embodiments are encompassed.

We claim:

1. An ophthalmic composition comprising a therapeutically effective amount of a non-steroidal anti-inflammatory agent (NSAID), a therapeutically effective amount of a steroidal anti-inflammatory, and an ophthalmically acceptable vehicle comprising a flowable mucoadhesive polymer, wherein the composition has a viscosity formulated for administration to the eye of a mammal in drop form.

2. The ophthalmic composition of claim 1, wherein said flowable mucoadhesive polymer is a lightly cross-linked carboxy-containing polymer.

3. The ophthalmic composition of claim 2, wherein said ophthalmically acceptable vehicle further comprises chitosan.

4. The ophthalmic composition of claim 3, wherein said chitosan is in a sufficient amount to allow said flowable mucoadhesive polymer to remain in suspension in the composition.

5. The ophthalmic composition according to claim 4, wherein said NSAID is present in a range from about 0.001% to about 1.0% by weight of the composition.

6. The ophthalmic composition according to claim 1, wherein NSAID is selected from the group consisting of: bromfenac, ketorolac, and nepafenac, and the steroidal anti-inflammatory is selected from the group consisting of dexamethasone, prednisolone, fluoromethalone, loteprednol etabonate, and difluprednate.

7. The ophthalmic composition according to claim 6, wherein the NSAID is nepafenac and the steroidal anti-inflammatory is dexamethasone.

8. The ophthalmic composition according to claim 1, wherein the steroidal anti-inflammatory is present in a range from about 0.01% to about 1% by weight of the composition.

9. The ophthalmic composition according to claim 1, wherein the composition has a pH of about 6.0 to about 8.5.

10. The ophthalmic composition according to claim 1, wherein the viscosity of the composition is in the range of about 1,000 to about 30,000 cps.

11. The ophthalmic composition according to claim 10, wherein the viscosity of the composition is in the range of about 1,000 to about 5,000 cps.

12. The ophthalmic composition according to claim 1, wherein the composition further comprises an additional therapeutically active agent selected from the group consisting of antibiotic agent, synthetic antibacterial agent, antifungal agent, synthetic antifungal agent, antineoplastic agent, anti-allergic agent, glaucoma-treating agent, antiviral agent and anti-mycotic agent.

13. A method for therapeutic treatment of an inflammatory condition of the eye or surrounding tissue in a mammal comprising steps of: (a) providing a non-steroidal anti-inflammatory agent (NSAID), a therapeutically effective amount of a steroidal anti-inflammatory, and an ophthalmically acceptable vehicle comprising a flowable mucoadhesive polymer, wherein the composition has a viscosity formulated for administration to the eye of a mammal in drop form; and (b) administering said composition to the eye of a mammal to treat inflammation or inflammatory conditions of the eye and/or surrounding tissue.

14. The method according to claim 13, wherein the NSAID is selected from the group consisting of: bromfenac, ketorolac, or nepafenac, and the steroidal anti-inflammatory and the steroidal anti-inflammatory is selected from the group consisting of dexamethasone, prednisolone, fluoromethalone, loteprednol etabonate, and difluprednate.

15. The method according to claim 13, wherein said ophthalmically acceptable vehicle further comprises chitosan.

16. The method according to claim 13, wherein said NSAID is present in a range from about 0.001% to about 1.0% by weight of the composition.

17. The method according to claim 13, wherein the steroidal anti-inflammatory is present in a range from about 0.01% to about 1% by weight of the composition.

18. The method according to claim 13, wherein the ophthalmic composition further comprises a therapeutically active agent selected from the group consisting of an antibiotic agent, a synthetic antibacterial agent, an antifungal agent, a synthetic antifungal agent, an antineoplastic agent, an anti-allergic agent, a glaucoma-treating agent, an antiviral agent and an anti-mycotic agent.

19. The method to claim 13, wherein the inflammatory condition is a retinal condition selected from the group consisting of: age related macular degeneration, AIDS-related ocular disease, CMV retinitis, birdshot retinochoroidopathy (BR), choroidal melanoma, coats disease, cotton wool spots, diabetic retinopathy diabetic macular edema, cystoid macular edema, lattice degeneration, macular disease, macular degeneration, hereditary macular dystrophy, macular edema, macular hole, macular pucker, central serous chorioretinopathy, ocular histoplasmosis syndrome (OHS), posterior vitreous detachment, retinal detachment, retinal artery obstruction, retinal vein occlusion, retinoblastoma, retinopathy of prematurity (ROP), retinitis pigmentosa, retinoschisis (acquired and x-linked), stargardt's disease, toxoplasmosis of retina and uveitis.

20. The method according to claim 19, wherein the inflammatory condition is cystoid macular edema.
